(19) United States
(12) Patent Application Publication
(10) Pub. No.: US 2007/0006877 A1
(43) Pub. Date: Jan. 11, 2007

(54) High efficiency inhaler adapter/nozzle/ancillary devices

(76) Inventors: Zongqin Zhang, Wakefield, RI (US); Jinbo Wang, Hubei (CN)

Correspondence Address: GAUTHIER & CONNORS, LLP, 225 FRANKLIN STREET, SUITE 2300, BOSTON, MA, 02110, US

(21) Appl. No.: 11/497,612

(22) Filed: Aug. 2, 2006

Related U.S. Application Data
(63) Continuation of application No. PCT/US05/10385, filed on Mar. 25, 2005.

(51) Int. Cl. A61M 11/00

(52) U.S. Cl. 128/200.14

(57) ABSTRACT

An inhaler nozzle configured to deliver a medicated aerosol to lungs of a user when the medicated aerosol is forced through said housing. The nozzle includes a housing having an entire length and an inner surface. The inner surface comprising a lower arcuate section and an upper section. The upper section has a first arcuate portion and a second arcuate portion. The first and second arcuate portions are positioned adjacent to each other to form a ridge. The lower arcuate section and the upper section can extend along at least a portion of the entire length of the housing.

### PRIORITY INFORMATION

This application claims priority to U.S. Provisional Patent Application No. 60/556,118 filed on Mar. 25, 2004.

### BACKGROUND OF THE INVENTION

1. Field of the Invention

This invention relates generally an inhaler nozzle used to deliver medicated aerosol to the lungs of the user. The nozzle is configured such that the medicated aerosol actually reaches the lungs rather than remaining in the user's mouth or laryngeal airway.

2. Description of the Prior Art

The American Lung Association estimated that 26 million Americans have been diagnosed with asthma in their lifetime. Of these 26 million Americans, 10.6 million have had an asthma episode in the past 12 months. An additional 4.9% of the US populations (about 13.9 million) have Chronic Obstructive Pulmonary Disease (COPD) in their lifetime. A metered-dose inhaler (MDI) and a dry-powder inhaler (DPI) are popular devices used in the treatment of these lung diseases. However, there are problems associated with inhalers which are well documented. Typically, only about 10% of aerosol medicine can be delivered to the lung region. The major problem associated with inhaler therapy is the massive aerosol deposition within oral cavity and on oropharyngeal airways. To minimize the problem and to deliver the aerosol drug in a more respirable form, inhalers having spacers and chambers of various designs have been developed. Some are as simple and straightforward as extension tubes placing the mouth at a greater distance from the inhaler. Others decelerate the aerosol by means of tortuous flow path routes or bluff body impact areas. While reducing the aerosol deposition at the back of throat, the current designs of existing spacers often contribute to a great drug loss within the delivery system. Additionally, most of the spacers are expensive and not easy to clean.

Effective delivery to a patient is a critical aspect of any successful drug therapy. Various routes of delivery exist. Oral drug delivery of pills, capsules and elixirs is perhaps the most convenient method, but many drugs are degraded in the digestive tract before they can be absorbed. Subcutaneous injection is frequently an effective route for systemic drug delivery, but enjoys a low patient acceptance. Aerosol therapy constitutes a major part of the therapeutic treatment for patients with lung disease such as asthma and bronchitis, and has potential for the system delivery of insulin, peptides and proteins as well. The MDI and DPI are popular devices used in aerosol therapies. Although these inhalers are safe, portable, multi-dose, and cost-effective ways to deliver inhaled medications, patients are known to experience problems with MDI and DPI as well. A survey conducted by a nationally recognized asthma research center indicated that only about 50% of the people who are now using MDI are using them “correctly”. The rest of the users may actually be getting as little as 1% of the prescribed medication delivered to their lungs. In fact, there is no clear defined “correct” inhalation procedure. A lot of doctors believe that the best way for patients to inhale medication is to use what's called the “open-mouth” method, which is very different from the “close-mouth” method that is prescribed by directions that come with the medication. Most of the directions were written 20 years ago when many asthma treatment medications were first introduced. However, not all doctors are persuaded that the open-mouth method is better for every patient. Meanwhile, new types of inhalers are being developed to replace the Freon-12 (the propellant in the old inhalers). Many of these inhalers are breath-activated, which are easier to use than old inhalers.

Numerous studies on aerosol medicine delivery have been published. Many investigators have examined the effects of aerosol diameter and flow rate on deposition of particles in the oral-pharyngeal-laryngeal airways. Deposition of monodisperse aerosol particles (2-12 microns) in the oral airway of healthy adult human volunteers has been reported. It is suggested that impaction is the dominant deposition mechanism for these cases. The deposition mechanism of ultrafine particles (<0.1 microns) in human oral airway replicas has also been studied. It has been shown that turbulent diffusional deposition is the dominant mechanism.

The distance between inhaler and mouth, shape and opening of the mouth, breath control, etc.), various inhaler and spacer design (i.e., cross-sectional geometry, spray angle, propellant pressure, etc.) will alter the particle's initial velocity, entrance position and angle, and therefore, results in distinct deposition patterns.

It has been determined that effects of mouthpiece diameter on deposition efficiency in an oral airway cast were significant and depend on particle size. It has been pointed out that the diameter may alter the airflow characteristics (i.e., air velocity and turbulence) for a given flow rate, leading to difference in deposition efficiency. In fact, the diameter will not only alter the airflow patterns, but also change the initial positions of inhaled aerosols relative to the oropharyngeal airway passage. It has been found that manipulating inhaler designs can have significant effects on aerosol delivery efficiency.

Considerable amount of research work has been done in the past, which has greatly advanced the technology of aerosol therapies and knowledge of particle depositions in human extrathoracic airways. However, it is worth noting that the efficiencies of current inhalers (with and without spacers) are excessively low. The database related to the aerosol therapy applications is especially inconclusive. Accordingly, more relevant R&D work is needed.

There is a great need for the development of an inhaler ancillary device that can significantly improve delivery efficiency by minimizing massive aerosol deposition at the back of throat, reducing the drug loses in the delivery system, and meanwhile cost less. It is noted that most medical insurance companies don't cover the cost of the spacer, since it is considered as ‘medical device”. This cost may be acceptable to patients having chronic conditions that require frequent use of inhaler medication for a long period of time, provided the patients are willing to frequently clean the devices. However, many patients need inhaler medications for only a short period of time, in which case the high cost of the spacer is very unsatisfactory. In addition, most spacers are too big to be put in a vest pocket and far too expensive to be considered disposable. There is a great need to develop a high efficiency, portable, light, reliable, inexpensive, disposable, adaptable and easy-to-use ancillary devices for use with various inhalers.

### SUMMARY OF THE INVENTION

The characteristics of respiratory flow and particle transportation patterns during aerosol therapy are important when developing a high efficiency, portable, light, reliable, inexpensive, disposable, adaptable and easy-to-use ancillary devices for use with various aerosol therapy system, especially for inhalers. This is accomplished by means of computer simulations and experimental investigations using extrathoracic airway models.

Aerosols emitted from various cross-sectional location of mouthpiece have different destinations as can be demonstrated in a computer simulation. The efficiency of drug delivery can be significantly improved if an ancillary device, such as a specially configured extended mouthpiece, is used to channel aerosols from where they are most likely to penetrate through the extrathoracic airways. This objective can be achieved by a good design of inhaler ancillary devices, including design of cross-sectional configuration, location and shape of air slit, outside diameter, tapered angle, and distance extended into the mouth. Aerosol delivery efficiency is defined as the number ratio of the aerosols that went through the extrathoracic airways to the aerosols that ejected from the inhaler.

An inhaler nozzle configured to deliver a medicated aerosol to lungs of a user when the medicated aerosol is forced through said housing. The nozzle has a housing having an entire length L and an inner surface. The inner surface comprising a lower arcuate section and an upper section. The upper section has a first arcuate portion and a second arcuate portion, wherein the first and second arcuate portions are positioned adjacent to each other to form a ridge. The lower arcuate section and the upper section can extend along at least a portion of the entire length L of the housing.

These and other features and objectives of the present invention will now be described in greater detail with reference to the accompanying drawings, wherein:

### BRIEF DESCRIPTION OF THE DRAWINGS

FIG. 1 is a breakaway perspective view of an embodiment of the invention;

FIG. 2A is a front end view of FIG. 1;

FIG. 2B is a sectional view FIG. 1 taken along lines 2B;

FIG. 2C is a rear end view of FIG. 1;

FIG. 3A is a front end view of an alternative embodiment of FIG. 1;

FIG. 3B is a sectional view of FIG. 3A taken along lines 3B;

FIG. 3C is a rear end view of FIG. 3A;

FIG. 4A is a front end view of yet another embodiment of FIG. 1;

FIG. 4B is a sectional view of FIG. 4A taken along lines 4B;

FIG. 4C is a rear end view of FIG. 4C;

FIG. 5A is a front end view of yet another embodiment of FIG. 1; and

FIG. 5B is a sectional view of FIG. 5A taken along lines 5B; and

FIG. 6A-D are cross sections of inhaler mouthpieces and aerosol delivery through the mouthpiece.

### DETAILED DESCRIPTION OF THE INVENTION

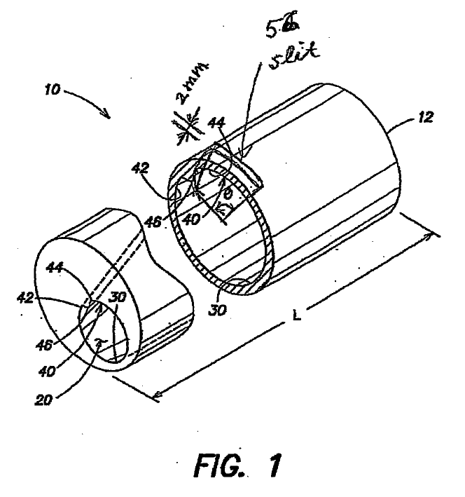

With reference to the figures an inhaler nozzle is shown at 10 and comprises a housing. FIG. 1 is a breakaway perspective view of an embodiment of the invention. The inner surface 20 of the housing 12 is configured to facilitate the delivery of a medicated aerosol to the lungs of a user when the medicated aerosol is forced through the housing 12. The inner surface 20 comprises a lower arcuate section 30 and upper section 40. The upper section 40 comprises a first arcuate portion 42 and a second arcuate portion 44. The first arcuate portion 42 and the second arcuate portion 44 are joined together to form a ridge 46. Both the lower arcuate section 30 and the upper section 40 can extend along the entire length L of the housing 12 or optionally can extend along at least a portion of the length L of the housing. Alternatively, the lower arcuate section 30 can extend along the entire length L of the housing and the upper section 40 can extend along at least a portion of the length L of the housing or vice versa. A slit 56 may be fabricated on the upper section 40. The width of the slit is within the rand of 1-5 mm, preferably 2 mm. The radian of the slit 56 is within the range of 15-180 degrees, preferably 60 degrees. The downward angle of the slit 56 is the same as the downward angle between upper section surface 30 and 40. The slit introduces a stream of air on the top of the inhaled aerosol medicine and to service as a layer of air cushion between the aerosol drug and throat and to prevent aerosol from contacting the surface of the inhaler.

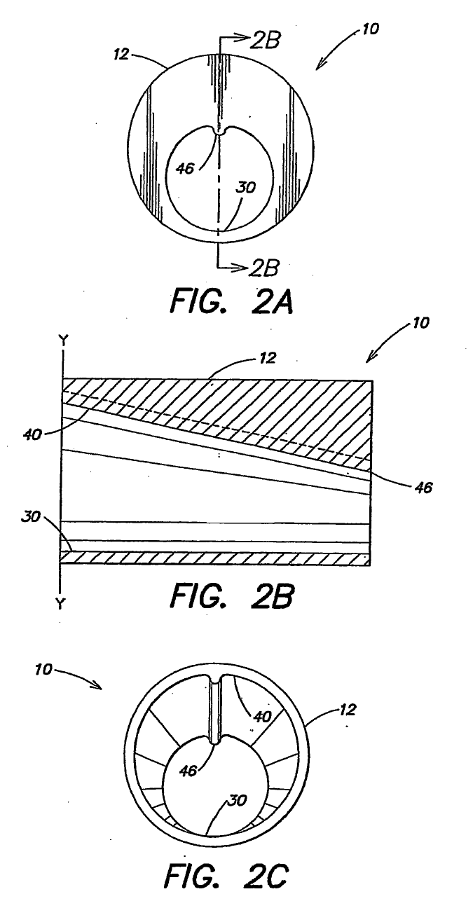

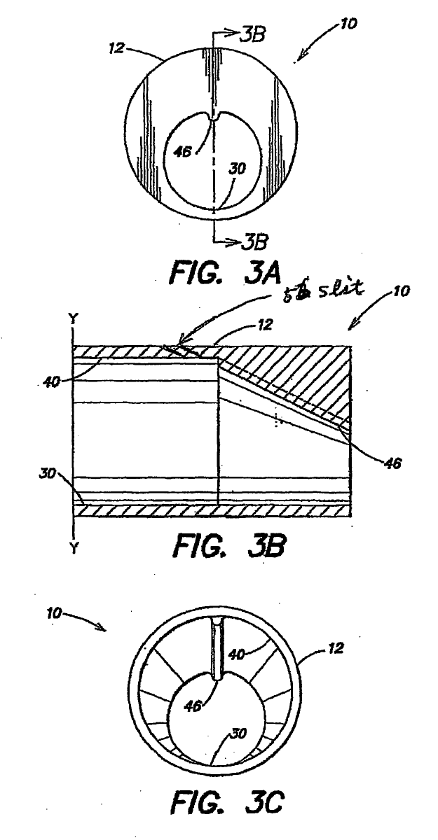

Referring to FIG. 2B, the upper section 40 extends downwardly along the entire length L of the inhaler nozzle 10 toward the lower arcuate section 30 at an angle within the range of between 5° to 40°, preferably 30°, to the vertical axis Y of the inhaler nozzle 10. FIG. 3B is a sectional view of FIG. 3A taken along lines 3B. Referring to FIG. 3B, the upper section 40 extends downwardly along about half the length L of the inhaler nozzle 10 toward the lower arcuate section 30 at an angle within the range of between 5° to 40°, preferably 30°, to the vertical axis Y of the inhaler nozzle 10.

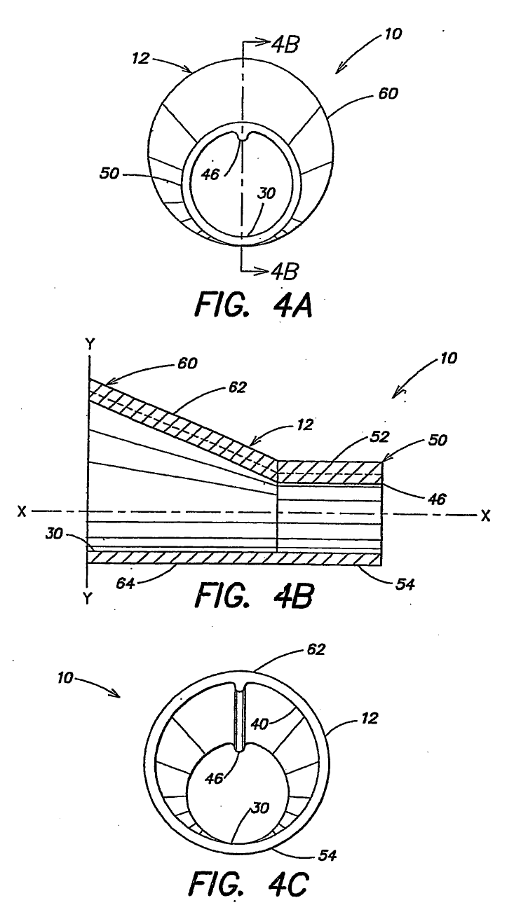

FIG. 4B is a sectional view of FIG. 4A taken along lines 4B, the housing 12 comprises a first segment 50 having a top and bottom surface 52, 54 and a second segment 60 having a top and bottom surface 62, 64. The top surface 62 extends upwardly from the top surface 52 at an angle within the range of between 5° to 40°, preferably 15°, from the horizontal axis X of the first segment 50.

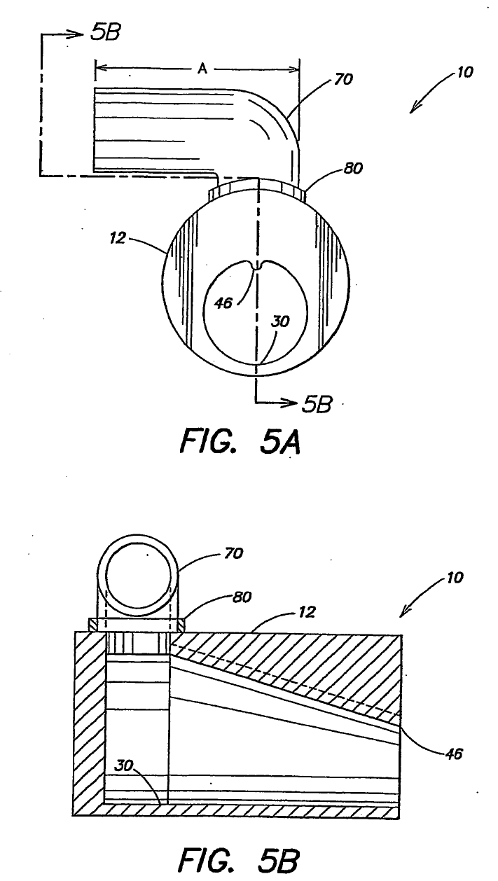

FIG. 5B is a sectional view of FIG. 5A taken along lines 5B. Referring to FIG. 5B, the inhaler nozzle 10 comprises a collar 80 which collar 80 is adapted to frictionally receive a tube 70 having a length A. The length A is positioned at a right angle to the length L inhaler nozzle 10.

Suitable moldable materials are used to construct the inhaler nozzle 10 and include moldable plastics.

The reduction of the aerosol deposition in extrathoracic airways, specifically, the oral-pharyngeal-laryngeal (OPL) airways is important. The OPL airways act as the first of a series of artificial filters that aerosol will encounter before reaching the targeted lower airway lung region. For propellant driven inhalers, aerosol velocities and sizes decrease drastically after the spray. Typically, the measured velocities are about 50 m/s at the nozzle orifice and close to 20 m/s when reach the back of throat. Thus, if aerosols can survive the OPL airways and enter thoracic region, their velocities and sizes are significantly reduced. A much smaller impaction force can be expected downstream of OPL. Therefore, the aerosol deposition is by the mechanism of inertial impaction, the dominant mechanism that cause massive aerosol deposition at the back of throat.

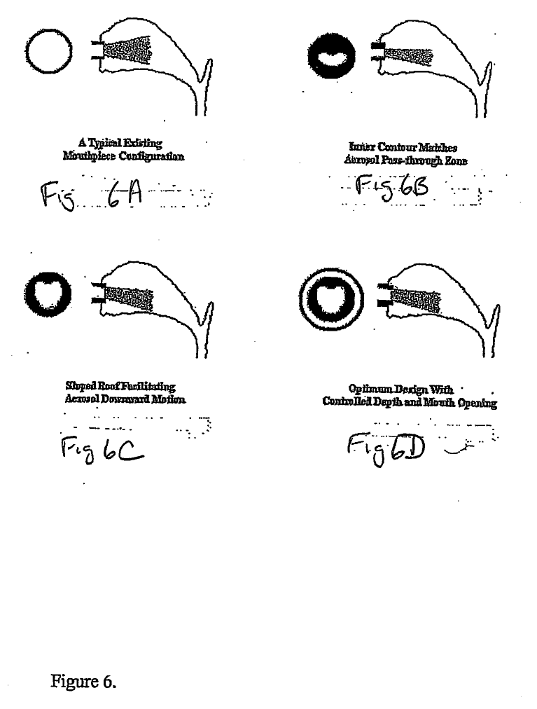

FIG. 6 illustrates the schematic drawings of various inhaler mouthpiece configurations including a typical existing design in FIG. 6A and new designs FIGS. B, C and D in accordance with the invention disclosed herein. ‘Close-mouth’ method, in which the lip encloses a mouthpiece, is used for the purpose of demonstration. The existing inhaler mouthpiece design has a circular or an oval cross sectional geometry with uniform tube thickness.

FIG. 6B demonstrates an improved cross sectional configuration design that improves aerosol delivery efficiency. The inner cross-sectional shape of the mouthpiece matches the contours of particle penetration zone revealed from computer simulation and experimental verification, while the outer configuration maintains the circular geometry for manufacture convenience or patient comfort, This design can be considered as a two-dimensional optimization: 1) it is a simple modification from the existing mouthpiece design where certain portions of cross-sectional (aerosols ejected from these portions are likely to be captured within the OPL airways) are blocked; 2) aerosols still enter the mouth in an horizontal direction as existing design; and 3) the design can be achieved by designing a 2-D optimum mouthpiece passage or simply adding a 2-D plastic plate (with the desired cross-sectional geometry) in front of the existing mouthpiece.

Further improvement of design is illustrated in FIG. 6. The mouthpiece has a sloped roof configuration facing the downward direction. When airflow exits the mouthpiece, the mainstream air levels out while the entrained aerosols near the top will continue their downward motions due to inertial forces. These are the same forces causing the aerosols deposited on the back of the throats. The downward angle will facilitate particles passing through the 90-degree downward bend of the oropharyngeal airway. Therefore, it will reduce the deposition at the back of throat caused by particle inertial impaction. This design is a three-dimensional optimization: 1) the inner passage can has a variable as well as a simple linearly sloped cross-sectional geometry, 2) aerosols enter the mouth in a downward direction, 3) this design can be achieved by design a 3-D optimum mouthpiece adaptor connected with most existing mouthpiece, 4) a simple version of the 3-D design can be achieved by placing a 2-D optimized plate configuration in front of a straight downward regular circular tube.

A preferred inhaler mouthpiece configuration is displayed in FIG. 6D. In this design, not only the aerosol initial position and angle, the inhaler distance extended into the oral cavity as well as the outer diameter of the mouthpiece is also optimized. It is noted that for most conventional designs, the distance of inhaler intruded into the mouth not only varies between patients to patients, but also changes between every therapy for the same individual. The outer diameter of a mouthpiece also plays an important role by controlling the opening of the mouth, therefore, affects the shape of oral cavity.

The main difference between the proposed device and the existing patents is that the existing patents are to slow down the flow and reduce the particle sizes into a respirable range while the goal of the design disclosed herein is to channel or guide the aerosols to the right location. It is an additional objective to develop a universal inhaler ancillary device. Essentially, this device is a hollow, flexible and extended mouthpiece with an optimum configuration. It will be designed to easily hook up to mouthpieces of most current inhalers, spacers and other aerosol delivery systems. The optimum mouthpiece configuration assures high efficiency. While extension places the inhaler at a greater distance from the mouth and thus further diffuse the aerosol velocity. The hollow and simple see-through structure minimizes the drug loses in the device and simplifies clean up. The new device is low cost, portable and deposable.

1. An inhaler nozzle configured to deliver a medicated aerosol to lungs of a user when the medicated aerosol is forced through said housing, said nozzle comprising:
a housing having an entire length and an inner surface, said inner surface comprising a lower arcuate section and an upper section, said upper section has a first arcuate portion and a second arcuate portion, the first and second arcuate portions are positioned adjacent to each other to form a ridge, said lower arcuate section and said upper section can extend along at least a portion of the entire length of the housing.

2. The inhaler nozzle of claim 1, wherein the lower arcuate section extends along the entire length of the housing and the upper section extends along at least a portion of the entire length of the housing.

3. The inhaler nozzle of claim 1, wherein the upper arcuate section extends along the entire length of the housing and the lower section extends along at least a portion of the entire length of the housing.

4. The inhaler nozzle of claim 1, wherein the upper section includes a slit.

5. The inhaler nozzle of claim 4, wherein the slit creates a layer of air cushion between the streaming of aerosols and the airway wall when aerosol is forced through the inhaler.

6. An adapter configured to receive an inhaler nozzle, the adapter having a housing having an entire length and an inner surface, said inner surface comprising a lower arcuate section and an upper section, said upper section has a first arcuate portion and a second arcuate portion, the first and second arcuate portions are positioned adjacent to each other to form a ridge, said lower arcuate section and said upper section can extend along at least a portion of the entire length of the housing.
